(19) United States
(12) Patent Application Publication
(10) Pub. No.: US 2003/0083717 A1
(43) Pub. Date: May 1, 2003

(54) Fuzzy inference machine

(75) Inventors: Michael Frank Mlynski, Aachen (DE); Walter Ameling, Aachen (DE); Max Schaldach, Erlangen (DE); Max Schaldach JR., Berlin (DE)

Correspondence Address: HAHN LOESER & PARKS, LLP, TWIN OAKS ESTATE, 1225 W. MARKET STREET, AKRON, OH, 44313, US

(73) Assignee: Biotronik Mess-und Therapiegeraete GmbH & Co Ingenieurbuero Berlin

(21) Appl. No.: 10/234,635

(22) Filed: Sep. 4, 2002

(30) Foreign Application Priority Data
Sep. 6, 2001 (DE) 101 44 441.9

(51) Int. Cl. A61N 1/08

(52) U.S. Cl. 607/59

(57) ABSTRACT

The invention is based on the concept of providing a fuzzy inference machine having a fuzzy logic unit. The fuzzy logic unit converts an input value into a fuzzy scalar on the basis of scalar fuzzy operators. The converted fuzzy scalars are processed by means of fuzzy interconnection operators to give scalar output values.

The invention is therefore based on providing the inference machine of an expert system with a fuzzy logic unit which can operate directly with scalar data values and which permits the processing of unsharp knowledge. Then neither a fuzzifier nor a defuzzifier are required. In accordance with the (unsharp) knowledge of the knowledge base, the input values are compared by (unsharp) fuzzy operators to the presettings and afford (unsharp) information in the range of from “completely false” to “completely true”.

Also shown is an electrical therapy device having a fuzzy inference machine of that kind.

The invention concerns a fuzzy inference machine and an electrical therapy device having a fuzzy inference machine.

With progress in technological development the therapy options in regard to implantable cardiac pacemakers and ICDs (implantable cardioverter defibrillators) have also improved. In that respect however the choice of the correct programming parameters for improving the condition of the patient is becoming increasingly more complicated, for the greater the amount of data that is collected in order to determine the optimum therapy, the correspondingly more laborious it becomes for the physician carrying out the treatment, to analyze and understand those data.

There are for example endeavors to provide systems which allow the physician carrying out the treatment, for example on the basis of expert systems, for diagnosis, analysis and choice of the appropriate therapy, to automatically investigate and interpret the collected data and to point the physician to unusual results.

U.S. Pat. No 5,713,938 discloses a fuzzy logic expert system for an implantable cardiac device. The implantable cardiac device in that case uses a knowledge-based expert system for the prognosis of a cardiac malfunction on the basis of operating parameters and/or patient characteristics. The fuzzy logic expert system receives numerical data from a cardiac pacemaker and fuzzifies those data in a fuzzy vector. A set of rules in then used to derive a diagnostic prognosis from the data. In that respect the rules represent an analysis which is effected by a treating physician and on the basis of which numerical values are converted into linguistic values and a diagnosis is thus executed using the knowledge, set down in the rules, in the treating physician.

An (expert) rule is initially composed of a condition and an action:

IF<condition>THEN<action>

Conditions can in turn be composed of sub-conditions which are linked to form the rule condition, that is to say for example:

IF<sub-condition 1>AND<sub-condition 2>THEN<action>

An inference machine which is intended to implement the conclusion mechanism must therefore effect evaluation of a sub-condition and interconnection of a plurality of sub-condition results.

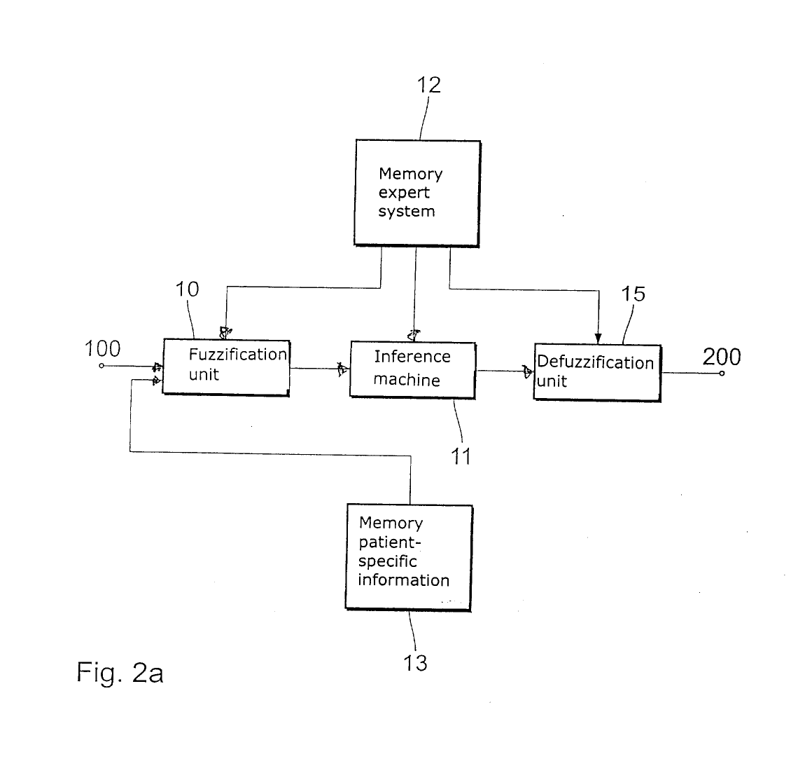

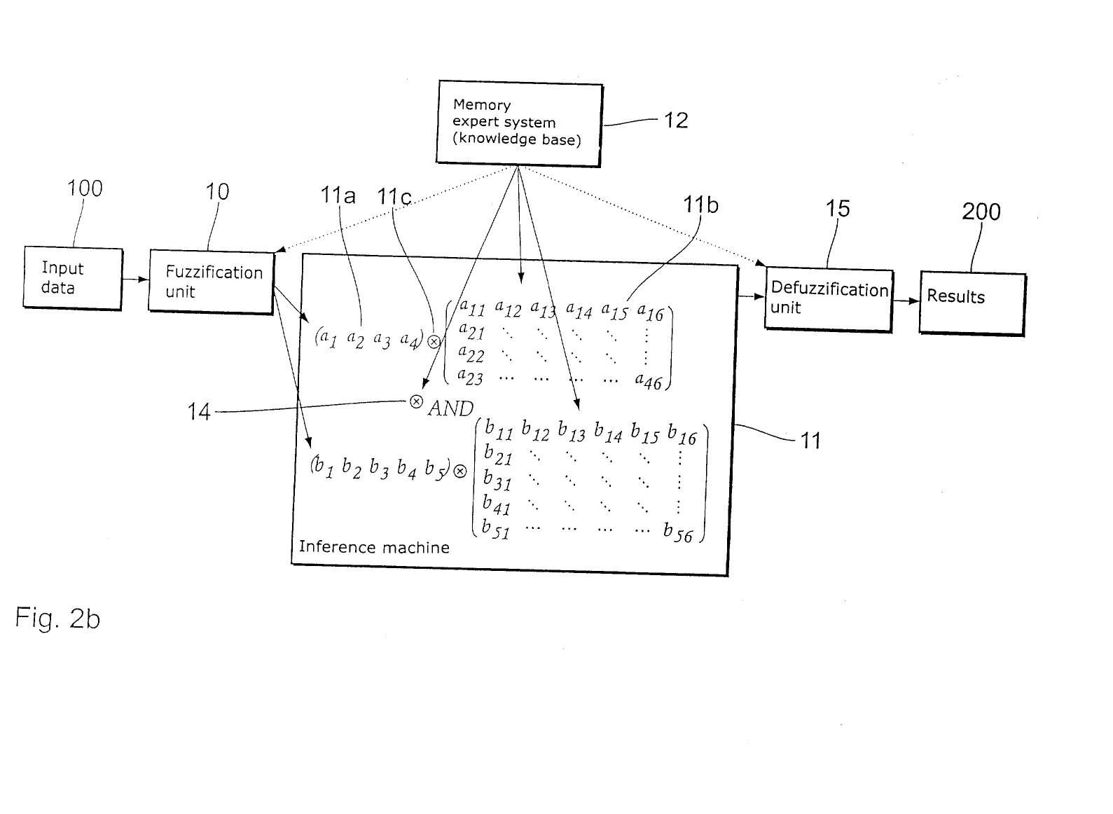

FIGS. 2a and b show a diagrammatic representation of a known fuzzy logic diagnosis system, FIG. 2b showing a conceptional representation of the diagnosis system and in particular the inference machine. A fuzzification unit 10 receives as input values 100 for example data from a cardiac pacemaker and implements fuzzification therewith. For fuzzification of the input values for example three association functions are defined and the input value is fuzzified or converted into a fuzzy vector on the basis of those association values. In an inference machine 11 the association values are fed to a logical interconnection, that is to say the procedure involves unsharp closure with unsharp amounts of values. In that respect, the fuzzy vector 11a which is fuzzified in the fuzzification unit 10 is multiplied by a fuzzy matrix (fuzzy relations) 11b in the inference machine 11, that multiplication representing a fuzzy cross-product 11c. The fuzzy matrices or the fuzzy relations 11b are set up on the basis of the expert system or expert knowledge stored in the expert system memory 12, that is to say on the basis of the rules and preset conditions. In the case of inference which takes place in the inference machine 11, not only the rules and preset conditions from the expert system but also patient-specific data from a memory for patient-specific items of information 13 are taken into account. It will be appreciated that those data also have to be initially fuzzified by the fuzzification unit 10 before they can be processed by the inference machine. The result vectors of the inferences in accordance with various sub-conditions are interconnected in an interconnection unit 14 (not shown) of the inference machine in accordance with the rules of the expert system. As the result of interconnection of the individual result vectors of the respective sub-conditions represents an unsharp quantity of values, those values have to be defuzzified so that they can be used for control or diagnosis purposes. That is then effected in a defuzzification unit 15.

The memory with the expert knowledge or the expert system 12 also accesses the fuzzification unit 10 as the number of defined fuzzy sets and the defined association functions are the stock of the knowledge base, that is to say the items of information as to how for example input data are to be fuzzified are not encoded in the fuzzification unit 10, but the fuzzification unit 10 receives those items of information from the knowledge base. The same correspondingly applies for the defuzzification unit 15.

In the fuzzification procedure a real input value is transformed into the degree of realization of the fuzzy sets. In dependence on the degree of realization of a fuzzy set a statement in that respect is deemed to be “completely false” (degree of realization 0) to “completely true” (degree of realization 1). The rules in the expert system are implemented by so-called fuzzy inference. The input value is fuzzified into a fuzzy vector and the preset conditions (for the rules) are set out in the form of fuzzy matrices or fuzzy relations. When using so-called conventional fuzzy logic however some problems emerge, which are briefly referred to hereinafter.

It is from time to time found to be difficult to implement textually formulated conditions (parts of conditions of rules), having regard to the defined fuzzy sets in fuzzy matrices. That implementation procedure often has to be executed manually as at the present time it is not possible to effect complete automation of that procedure.

The computing expenditure for evaluation of a rule which is implemented by the fuzzy sets and fuzzy matrices rises with the number of fuzzy sets. As a certain number of fuzzy sets is absolutely necessary for rational implementation of fuzzy rules, that results in a high level of computing expenditure. Particularly when dealing with knowledge-based systems with a knowledge base of several hundreds to some thousands of rules the level of computing expenditure required is sometimes no longer a viable proposition.

When using generally acknowledged defuzzification methods (keypoint methods or approximate keypoint methods), the problem still remains that a finite quantity of fuzzy sets is projected onto a continuous range. That however gives discontinuities in higher derivatives of the initial function, which is represented in the results space in the form of “step-like” configurations.

If the defined fuzzy sets of a linguistic variable are further subsequently altered, for example because the instantaneously defined fuzzy sets are not sufficient or are inadequate for a new rule which is to be incorporated into the knowledge base, then all rules which have already been implemented or to put it more accurately the relational matrices in which that linguistic variable is used have to be checked and possibly adapted.

Therefore the object of the invention is to provide a fuzzy inference machine which reduces the level of computing expenditure in comparison with known inference machines and which at the same time permits simplified implementation of the rules set up.

That object is attained by a fuzzy inference machine as set forth by the features of accompanying claim 1.

In this respect the invention is based on the notion of providing a fuzzy inference machine having a fuzzy logic unit. The fuzzy logic unit in this case converts an input value into a fuzzy scalar on the basis of scalar fuzzy operators. The converted fuzzy scalars are processed to form scalar output values by means of fuzzy interconnection operators.

This has the advantage in particular that fuzzy processing is now effected in only two stages, while fuzzy processing in accordance with the state of the art is effected in four stages—fuzzification, inference, interconnection of the inference results and defuzzification. Defuzzification is eliminated here as the output value of fuzzy processing is a scalar. The fact that both the operators and also the parameters and data in the inference machine are scalar—instead of being vectors or matrices as is the case in the state of the art—affords a substantial reduction in the level of computing expenditure.

In a further embodiment of the invention the fuzzy logic unit has a scalar fuzzy comparison unit. In the scalar comparison unit a scalar fuzzy comparison operator is applied as a fuzzy operator to an input value. In that case the deviation of the input value from a predetermined threshold value represents an indication of equality of the input value and the threshold value. In this case the result of the scalar fuzzy comparison operator is 1 if the input value and the threshold value are identical. The result of the scalar fuzzy comparison operator tends towards zero if the magnitude of the difference between the input value and the threshold value goes towards infinity.

In a further configuration of the invention the fuzzy logic unit has a scalar fuzzy comparison unit. A scalar fuzzy comparison operator as a fuzzy operator is applied to an input value, wherein the magnitude of the deviation of the input value from a predetermined threshold value provides an indication of inequality of the input value and the threshold value. The result of the scalar fuzzy comparison operator is 0 if the input value and the threshold value are identical. The result of the scalar fuzzy comparison operator tends towards 1 if the magnitude of the difference between the input value and the threshold value goes towards infinity.

In a further embodiment of the invention the fuzzy logic unit has a scalar fuzzy comparison unit in which a scalar fuzzy comparison operator is applied as a fuzzy operator to an input value, wherein the magnitude of the difference between the input value and a predetermined threshold value provides an indication of whether the input value is lower than the threshold value. The result of the scalar fuzzy comparison operator is 0.5 if the input value and the predetermined threshold value are identical. The result of the scalar fuzzy comparison operator tends towards 1 if the difference between the threshold value and the input value tends towards infinity and the result of the scalar fuzzy comparison operator tends towards 0 if the difference between the input value and the threshold value tends towards infinity.

In a further embodiment of the invention the fuzzy logic unit has a scalar fuzzy comparison unit in which a scalar fuzzy comparison operator is applied as a fuzzy operator to an input value, wherein the magnitude of the difference between the input value and a predetermined threshold value provides an indication of whether the input value is larger than the threshold value. The result of the scalar fuzzy comparison operator is 0.5 if the input value and the predetermined threshold value are identical. The result of the scalar fuzzy comparison operator tends towards 0 if the difference between the threshold value and the input value tends towards infinity and the result of the scalar fuzzy comparison operator tends towards 1 if the difference between the input value and the threshold value tends towards infinity.

The advantages of the configurations of the invention which are described in the last four paragraphs are in particular that the application of a scalar fuzzy comparison operator to a real input value results in a fuzzy scalar as the result of the comparison operator. While, in an inference machine in accordance with the state of the art, fuzzification of the input values has to be effected first and then an inference has to be formed, in these embodiments of the invention it is only necessary to apply a comparison operator to the input value or values. Application of one of the comparison operators replaces fuzzification of the input values and formation of the inference including the vector and matrix calculation procedure required for that purpose.

In a preferred configuration of the invention the scalar fuzzy comparison unit has a memory in which an operational parameter can be stored. The form of the transition of the result of the comparison operator from 0 to 1 or vice-versa can be influenced on the basis of the operational parameter. By varying the transition parameter it is possible to alter the degree of “unsharpness”, that is to say the form of the transition from 0 to 1 for the result of the operation.

The object of the invention is further attained by an electrical therapy device having the features of accompanying claim 7.

In this respect the invention is based on the notion of evaluating cardiac events on the basis of a fuzzy inference machine in an electrical therapy device and possibly effecting a diagnosis.

The use of a fuzzy inference machine in a pacemaker, ICD or comparable implantable therapy systems is found to be advantageous as such a fuzzy inference machine is suitable for implementing the rules of an expert system without in that respect the level of computing expenditure being excessively high.

Embodiments of the invention are described hereinafter with reference to the accompanying drawing in which:

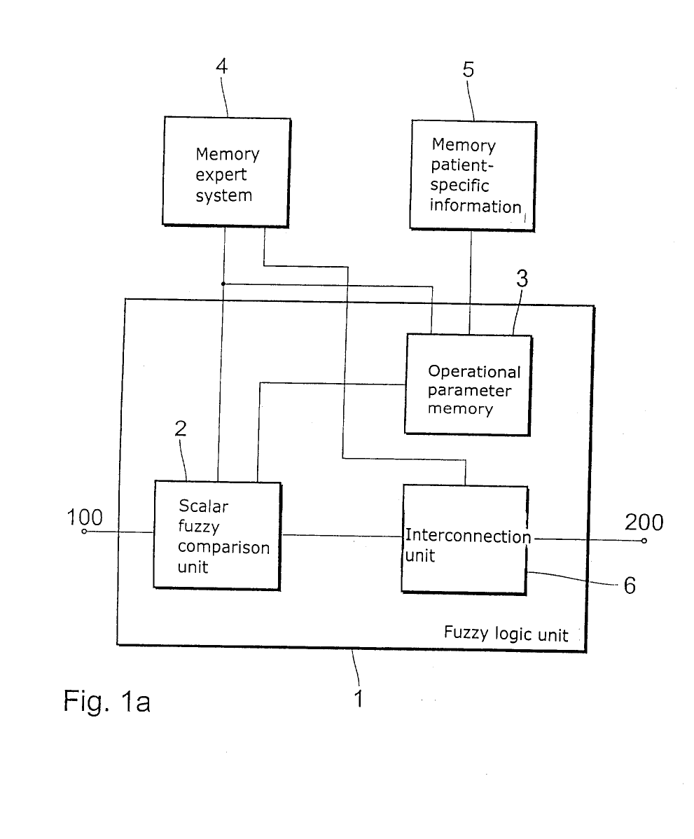

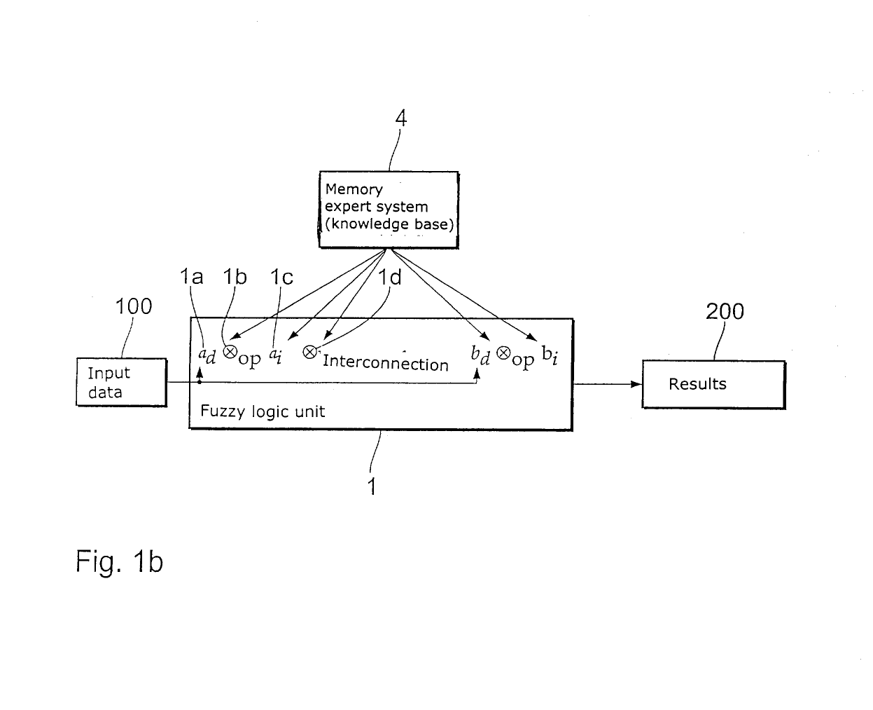

FIGS. 1a and b show a fuzzy logic diagnosis system with a fuzzy inference machine in accordance with an embodiment of the invention, and

FIGS. 2a and b show a fuzzy logic diagnosis system in accordance with the state of the art.

FIG. 1a, b shows a fuzzy logic diagnosis system in accordance with an embodiment of the invention, FIG. 1b showing a conceptional representation of the fuzzy logic unit 1 as a comparison with the conceptional representation of the known inference machine shown in FIG. 2b. The fuzzy inference machine has a fuzzy logic unit 1, an expert system memory 4 and a memory for patient-specific information 5, the fuzzy logic unit 1 being respectively connected to the memory 4 and the memory 5. The fuzzy logic unit 1 has a scalar fuzzy comparison unit 2, an interconnection unit 6 and an operational parameter memory 3. The expert system memory 4 is connected to the interconnection unit 6. The scalar fuzzy comparison unit 2 is connected at its input side to the output 100 of a pacemaker, the expert system memory 4 and the operational parameter memory 3 and at the output side to the interconnection unit 6 which in turn at its output side outputs the results 200 of the inference. The operational parameter memory 3 is connected at the input side to the expert system memory 4 and the memory for patient-specific information 5 and at the output side to the scalar fuzzy comparison unit 2.

The fuzzy logic unit 1 receives data from a pacemaker as input values 100. That input value is converted by means of fuzzy comparison operators into a fuzzy scalar in the scalar fuzzy comparison unit 2. The degree of “unsharpness” of the result of the operation can be influenced on the basis of the operational parameters stored in the memory 3. The result of the calculation for example of a sub-condition in the fuzzy logic unit 1 represents a fuzzy scalar. If there are a plurality of sub-conditions 1a and they are to be interlinked, the results of those sub-conditions, that is to say the output values of the scalar fuzzy comparison unit 2 are interconnected, as indicated at 1d, to the respective input values of the interconnection unit 6 and in accordance with the rules of the expert system stored in the memory 4. The output values of the interconnection unit 6 are scalar values and therefore do not have to be defuzzified.

Based on the need to be able to mathematically determine textual expressions or conditions such as for example:

“IF a value is fairly high THEN . . .” or

“IF a value is very high THEN . . .”without in that respect having to introduce multi-dimensional variables or vectors, or matrices, fresh operators are introduced. Those operators serve to convert the conditions of a rule from an expert system directly into a form which can be calculated mathematically and thus by an algorithm.

For example four comparison operators can be realized by means of the scalar fuzzy comparison unit 2:

a) In the case of the operator FE (Fuzzy Equal), a first and a second operand are compared in terms of their equality. If the first operand is identical to the second then the result of the operator FE is equal to 1, that is to say the condition is completely true. If the magnitude of the difference between the first and second operands tends towards infinite then the result of the operator FE tends towards 0, that is to say the condition is completely false.

b) The operator FNE (Fuzzy Not Equal) compares a first and a second operand in respect of their inequality. If the first operand is equal to the second then the result of the operator FNE is equal to 0, that is to say the condition is completely false. If the magnitude of the difference between the first and second operands tends towards infinite then the result of the operator FNE tends towards 1, that is to say the condition is completely true.

c) The operator FL (Fuzzy Less) compares the two operands in order to establish whether the first operand is smaller than the second operand. If the first operand is equal to the second operand then the result of the operator FL corresponds to 0.5, that is to say the condition is neither true nor false. If the difference between the second and the first operands tends towards infinite then the result of the operator FL tends towards 1, that is to say the condition is completely true. If the difference between the first and second operands tends towards infinite the result of the operator FL tends towards 0, that is to say the condition is completely false.

d) The operator FG (Fuzzy Greater) compares the two operands in order to ascertain to what extent the first operand is greater than the second operand. If the first operand is identical to the second then the result of the operator FG gives 0.5, that is to say the condition is neither true nor false. If the difference between the second and the first operands tends towards infinite then the result of the operator FG tends towards 0, that is to say the condition is completely false. If the difference between the first and second operands tends towards infinite then the result of the operator FG tends towards 1, that is to say the condition is completely true.

The result of a condition ascertained by means of the fuzzy logic unit 1 is not only a scalar but in addition is always of a value of between 0 and 1. In that respect 0 is to be interpreted in the Boolean sense as “completely false” and 1 is to be interpreted in the Boolean sense as “completely true”. All values between 0 and 1 thus form unsharp intermediate statements, how for example 0.5, that is to say unknown, cannot be decided.

In regard to all the above-indicated operators the degree of “unsharpness”, that is to say the form of the transition from 0 to 1 or vice-versa, of the result of the comparison operation can be adjusted by varying the operational parameter.

In order to be able to interconnect a plurality of sub-results, various interconnecting operators are used in the interconnection unit 6. For that purpose it is possible to use Boolean operators or also so-called compensatory operators. The latter are distinguished in that, in a situation involving interconnection of many sub-results, in regard to which there is a marked majority in regard to a statement, that majority is taken into account in every case.

Implementation of a rule of the expert system or expert knowledge stored in the memory 4 is effected by applying one of the four above-listed comparison operators with a corresponding operational parameter in order to ascertain the results of sub-conditions of the rule. The various results of the sub-conditions are then interconnected on the basis of the presettings of the corresponding rule in the comparison unit 6 on the basis of predetermined interconnection operators.

The fuzzy inference machine can be used for example in a risk analyzer for ascertaining and diagnosing intra-cardiac conditions which threaten the well-being of a patient. For that purpose an intra-cardiac ECG signal is recorded by means of a sense electrode and an electrode line. The recorded ECG signal is fed to a detection unit in which the QRS-complexes, the morphology of the QRS-complexes, the corresponding intervals and various kinds of extrasystoles and other ECG parameters are detected.

In addition an expert system is provided, in which the occurrence of given QRS complexes and other ECG parameters are correlated with a diagnosis, that is to say the rule base for the expert system is set up. It will be appreciated in that respect that it is possible to have recourse to the experience and knowledge of a large number of physicians and this can be incorporated into the expert system. When using the above-specified comparison operators it is possible for textually formulated rules to be easily converted into mathematical formulations so that production of the rule base and the conversion thereof into and the implementation thereof in mathematical formulations which can be easily calculated by algorithms is possible without major difficulties.

The fuzzy inference machine and the rule base to be implemented can serve for example to ascertain the risk of sudden heart death. For that purpose a QRS complex is detected as described above. It is then necessary to establish whether and which ventricular extrasystoles (VES) are present and which are suitable for analysis. The VES is investigated in respect of given criteria such as for example the time interval. Then the sum of all suitable VES is formed and a corresponding time interval is ascertained. Further parameters such as for example age and the question of whether the situation involves an infarction patient are also incorporated into the assessment.

The present fuzzy inference machine is suitable for use both in terms of diagnostic of given cardiac events and also in terms of control of the pacing or defibrillation function of electrical therapy units such as for example cardiac pacemakers or defibrillators. It will be appreciated that it is also possible to involve a combination of detection or diagnosis of cardiac events with subsequent control of the appropriate counter-measures by the fuzzy inference machine set forth herein.

Diagnosis or control which effected by the fuzzy inference machine depends solely on the given expert system and the corresponding rule basis. Diagnosis can be effected in situ or the ECG data can be stored over a predetermined period of time in an electrical therapy unit in order then to be read out for example telemetrically so that they are analyzed in an extra-corporal device by the fuzzy inference machine and a suitable diagnosis is produced.

The invention is therefore based on providing the inference machine of an expert system with a fuzzy logic unit which can operate directly with scalar data values and which permits the processing of unsharp knowledge. Then neither a fuzzifier nor a defuzzifier are required. In accordance with the (unsharp) knowledge of the knowledge base, the input values are compared by (unsharp) fuzzy operators to the presettings and afford (unsharp) information in the range of from “completely false” to “completely true”.

1. A fuzzy inference machine having a fuzzy logic unit (1) which is adapted to convert an input value into a fuzzy scalar by means of scalar fuzzy operators, wherein fuzzy scalars are processed by means of fuzzy interconnection operators to form scalar output values.

2. A fuzzy inference machine as set forth in claim 1 characterized in that the fuzzy logic unit (1) includes a scalar fuzzy comparison unit (2) which is adapted to apply a scalar fuzzy comparison operator as a fuzzy operator to an input value in such a way that the magnitude of the deviation of the input value from a predetermined threshold value provides an indication of the equality of the input value and the threshold value, wherein the result of the scalar fuzzy comparison operator is 1 if the input value and the threshold value are identical and wherein the result of the scalar fuzzy comparison operator tends towards 0 if the magnitude of the difference between the input value and the threshold value tends towards infinite.

3. A fuzzy inference machine as set forth in claim 1 characterized in that the fuzzy logic unit (1) includes a scalar fuzzy comparison unit (2) which is adapted to apply a scalar fuzzy comparison operator as a fuzzy operator to an input value in such a way that the magnitude of the deviation of the input value from a predetermined threshold value provides an indication of the inequality of the input value and the threshold value, wherein the result of the scalar fuzzy comparison operator is 0 if the input value and the threshold value are identical and wherein the result of the scalar fuzzy comparison operator tends towards 1 if the magnitude of the difference between the input value and the threshold value tends towards infinite.

4. A fuzzy inference machine as set forth in claim 1 characterized in that the fuzzy logic unit (1) includes a scalar fuzzy comparison unit (2) which is adapted to apply a scalar fuzzy comparison operator as a fuzzy operator to an input value in such a way that the magnitude of the difference between the input value and a predetermined threshold value provides an indication of whether the input value is less than the threshold value, wherein the result of the scalar fuzzy comparison operator is 0.5 when the input value and the predetermined threshold value are identical, wherein the result of the scalar fuzzy comparison operator tends towards 1 when the difference of the threshold value and the input value tends towards infinite and wherein the result of the scalar fuzzy comparison operator tends towards 0 when the difference of the input value and the threshold value tends towards infinite.

5. A fuzzy inference machine as set forth in claim 1 characterized in that the fuzzy logic unit (1) includes a scalar fuzzy comparison unit (2) which is adapted to apply a scalar fuzzy comparison operator as a fuzzy operator to an input value in such a way that the magnitude of the difference between the input value and a predetermined threshold value provides an indication of whether the input value is greater than the threshold value, wherein the result of the scalar fuzzy comparison operator is 0.5 when the input value and the predetermined threshold value are identical, wherein the result of the scalar fuzzy comparison operator tends towards 0 when the difference of the threshold value and the input value tends towards infinite and wherein the result of the scalar fuzzy comparison operator tends towards 1 when the difference of the input value and the threshold value tends towards infinite.

6. A fuzzy inference machine as set forth in one of the preceding claims characterized in that the fuzzy logic unit (1) has a memory (3) in which an operational parameter is stored, wherein the form of the transition of the result of the respective comparison operator from 0 to 1 or vice-versa can be adjusted on the basis of said operational parameter.

7. An electrical therapy device having an inference machine as set forth in one of claims claim 1 through 6.

8. An electrical therapy device as set forth in claim 7 characterized by an expert system memory (4) which is adapted to store expert knowledge, and a memory for patient-specific information (5) which is adapted to store patient-specific data.
